(19) United States
(12) Patent Application Publication
(10) Pub. No.: US 2014/0025173 A1
(43) Pub. Date: Jan. 23, 2014

(54) GLENOID IMPLANTS HAVING ADJUSTABLE BASE PLATES

(71) Applicant: TORNIER ORTHOPEDICS IRELAND LTD., Macroom (IE)

(72) Inventors: Jean-Emmanuel CARDON, Biviers (FR); John FENLIN, Bryn Mawr, PA (US); Gregory P. NICHOLSON, Western Springs, IL (US); Wayne BURKHEAD, Dallas, TX (US)

(21) Appl. No.: 13/949,032

(22) Filed: Jul. 23, 2013

Related U.S. Application Data
(60) Provisional application No. 61/674,702, filed on Jul. 23, 2012.

(30) Foreign Application Priority Data
Sep. 18, 2012 (FR) 1258748

(51) Int. Cl. A61F 2/40

(52) U.S. Cl. CPC A61F 2/4081
USPC 623/19.13

(57) ABSTRACT

A glenoid implant for a shoulder prosthesis is adapted to be implanted in the glenoid of a patient. The glenoid implant includes an articular body for articulating the glenoidal implant with a humeral component. A plate supports the articular body. An anchoring mechanism for anchoring the glenoid implant along an anchoring axis is adapted to be secured to the glenoid. A securing mechanism secures the plate in position relative to the anchoring mechanism and the glenoid. An adjustment mechanism facilitates selectively adjusting the position of the plate, before it is secured in position by the securing mechanism, in rotation about the anchoring axis and in translation transversely relative to the anchoring axis.

### CROSS-REFERENCE TO RELATED APPLICATION

This application claims the benefit of U.S. Provisional Patent Application Ser. No. 61/674,702, filed on Jul. 23, 2012, and French Patent Application No. 1258748, filed on Sep. 18, 2012, which are hereby incorporated by reference in their entirety.

### TECHNICAL FIELD

The present invention relates to glenoid implants for shoulder prostheses. The invention also relates to support plates for articular bodies suitable for being provided in such glenoid implants. The invention also relates to surgical kits including such glenoid implants.

### BACKGROUND

A glenoid implant typically includes an articular body that is adapted to articulate with the head of a humeral component (for example, the anatomical head of the humerus or a portion of a humeral implant). In some cases, the glenoid implant may provide an anatomical configuration in which the articular body includes a cavity which replaces the glenoid cavity. In other cases, the glenoid implant may provide a reversed configuration in which the articular body includes a hemispherical dome that cooperates with a complementary cavity defined by a humeral implant. The articular body may include metal, for example, a titanium alloy, ceramic material, or a synthetic material, for example, polyethylene.

For some designs, an implant includes an insert for fixing the articular body to the glenoid. The insert is permanently secured in the glenoid by the surgeon before the articular body is positioned on the insert. The implant also includes means for securing the rotation of the insert and the articular body in relation to the glenoid. In some cases, the glenoid implant needs to be replaced. The insert remains coupled to the glenoid and the articular body is replaced. The surgeon may provide, with relative difficulty, a new articular body of a different configuration. For example, the implant may be converted from an anatomical configuration to a reversed configuration. Furthermore, if the implant is to be converted from an anatomical configuration to a reversed configuration, the glenoid dome must typically be offset toward an inferior portion of the glenoid relative to the position previously defined by the insert. In other words, the articular center for a glenoid component having an anatomical configuration may be different from the articular center for a glenoid component having a reversed configuration. As such, ranges of articular bodies are available which have different offsets of the articular surface. However, the articular bodies are expensive and therefore this solution is not completely satisfactory.

### SUMMARY

In some embodiments, an object of the present invention is to provide an improved glenoid implant, particularly in terms of versatility, simplicity, and cost of implementation.

In some embodiments, the invention relates to a glenoid implant for a shoulder prosthesis. The glenoid implant is adapted to be implanted in the glenoid of a patient. The glenoid component includes an articular body for articulating the glenoid implant with a humeral component. A plate supports the articular body. An anchoring mechanism anchors the glenoid component along an anchoring axis and is adapted to be secured to the glenoid. A securing mechanism secures the plate in position relative to the anchoring mechanism and the glenoid. An adjustment mechanism facilitates selectively adjusting the position of the plate, before it is locked in position by the securing mechanism, in rotation about the anchoring axis and in translation transversely relative to the anchoring axis.

In some embodiments, the invention improves the versatility, the simplicity, and the cost of implementing a glenoid implant, which may have an anatomical or reversed configuration. In some embodiments, the anchoring mechanism may remain implanted in the glenoid to facilitate later revision of the shoulder prosthesis. In some embodiments, the adjustment mechanism facilitates accurate positioning of the plate and, thus, the articular body relative to the anchoring mechanism and the glenoid. In some embodiments, the offset of the articular body relative to the anchoring mechanism, and thus the glenoid, is adjusted via the plate and the adjustment mechanism; as such, a variety of potentially expensive articular bodies are not needed. In some embodiments, the plate may be rotatably and transversely translatably adjusted to occupy a desired position based on the anatomical dimensions of the shoulder of the patient. In some embodiments, the plate may be pivotably or tiltably adjusted relative to the anchoring axis if the surface of the glenoid is not perpendicular to the anchoring axis. In some embodiments, implants according to the invention are modular and convertible. In some embodiments, implants according to the invention are provided as part of a surgical kit.

In some embodiments, the adjustment mechanism includes a ball-type connection that is adapted to facilitate adjusting the position of the plate, before it is locked in position by the securing mechanism, tiltably relative to the anchoring axis. In some embodiments, the adjustment mechanism includes a groove formed in the plate. The groove extends in a direction transverse to the anchoring axis and facilitates adjustment of the position of the plate transversely to the anchoring axis and independently of rotation of the plate about the anchoring axis. In some embodiments, the groove includes at least two cavities that each define a distinct position of the plate transverse to the anchoring axis and independently of rotation of the plate about the anchoring axis. In some embodiments, the adjustment mechanism includes a pin that cooperates with the anchoring mechanism and the plate to adjust the position of the plate relative to the anchoring axis. In some embodiments, the articular body has one of an anatomic and a reversed configuration.

In some embodiments, the invention provides a plate for supporting an articular body. Such a plate is part of a glenoid implant as described above.

In some embodiments, the invention relates to a glenoid implant for a shoulder prosthesis. The glenoid implant is adapted to be implanted in the glenoid of a patient. The glenoid implant includes an anchor adapted to be secured to the glenoid. The anchor defines an anchoring axis. A base includes a slot adapted to relatively movably receive the anchor. The slot and the anchor facilitate rotational adjustment of the base relative to the anchor about the anchoring axis and translational adjustment of the base relative to the anchor in a direction transverse to the anchoring axis. An articular body is adapted to be supported by the base and to articulate with a humeral component.

In some embodiments, the invention provides a surgical kit that includes one or more glenoid implants for a shoulder prosthesis (for example, including a glenoid implant as described above). The glenoid implant is adapted to be implanted in the glenoid of a patient. The kit includes at least one articular body for articulation of the glenoid implant with a humeral component. The kit further includes at least one plate for supporting the articular body. The kit includes an anchoring mechanism that defines an anchoring axis and is adapted to be secured to the glenoid. The kit includes a securing mechanism for securing the position of the plate relative to the anchoring mechanism and the glenoid. The kit further includes an adjustment mechanism for selectively adjusting the position of the at least one plate, before it is locked in position by the securing mechanism, in rotation about the anchoring axis and transversely relative to the anchoring axis.

In some embodiments, the adjustment mechanism includes a groove formed in the at least one of the plate along a direction transverse to the anchoring axis. The groove facilitates adjusting the position of the plate transversely relative to the anchoring axis independently of rotation of the plate about the anchoring axis. In some embodiments, the kit includes at least two selectively adjustable plates. The plates are selectively adjustable to different configurations in terms of rotation about the anchoring axis and transversely relative to the anchoring axis.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is an exploded perspective view of a glenoid implant and a plate of the glenoid implant according to embodiments of the present invention;

FIG. 2 is another exploded perspective view of the glenoid implant and the plate of FIG. 1;

FIG. 3 is a partial section view of the glenoid implant of FIG. 1 implanted in the glenoid of a patient;

FIG. 4 is a front elevation view of the plate of FIG. 1;

FIG. 5 is a side view of a pin of the glenoid implant of FIG. 1;

FIG. 6 is a partial perspective view of the glenoid implant of FIG. 1 in an assembled configuration;

FIG. 7 is a view of the glenoid implant along the arrow VII in FIG. 6;

FIG. 8 is a front elevation view of a glenoid implant plate according to embodiments of the present invention;

FIG. 9 is a front elevation view of a glenoid implant plate according to embodiments of the present invention;

FIG. 10 is a front elevation view of a glenoid implant plate according to embodiments of the present invention;

FIG. 11 is a front elevation view of a glenoid implant plate suitable for equipping a surgical kit according to embodiments of the present invention;

FIG. 12 is a perspective view of the plate of FIG. 11;

FIG. 13 is a front elevation view of a glenoid implant plate suitable for equipping a surgical kit according to embodiments of the present invention; and

FIG. 14 is a perspective view of the plate of FIG. 13.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

This application incorporates by reference U.S. patent application Ser. No. 13/363,159, filed on Jan. 31, 2012, U.S. Provisional Patent Application No. 61/1,438,570, filed on Feb. 1, 2011, and French Patent Application No. 20110050994, filed on Feb. 8, 2011, for all purposes.

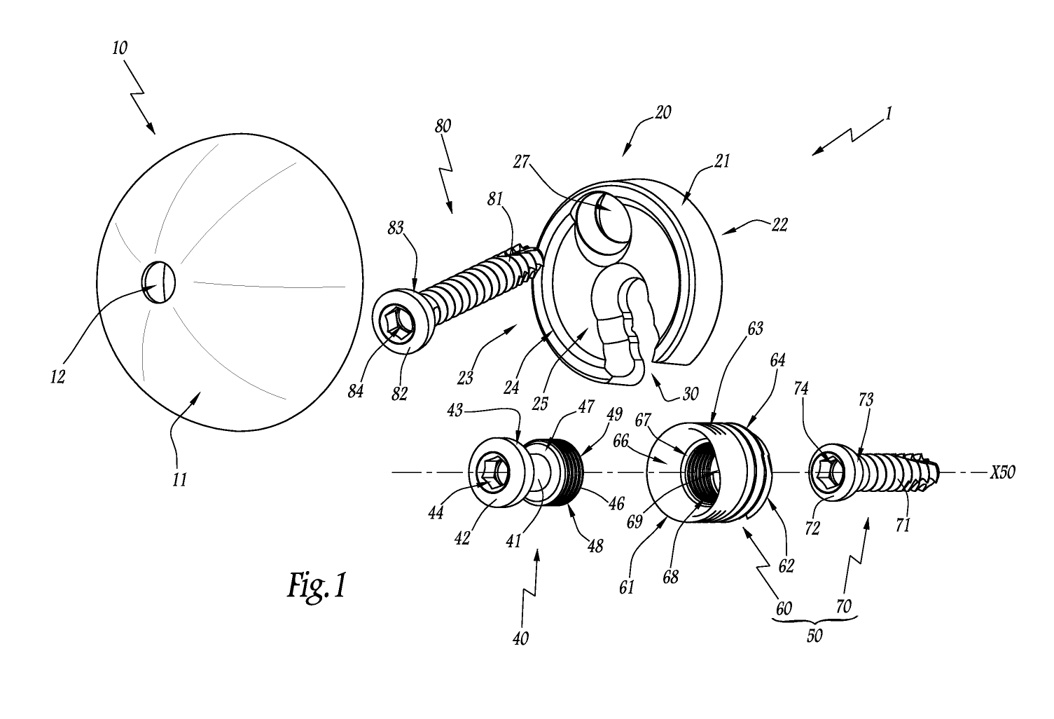

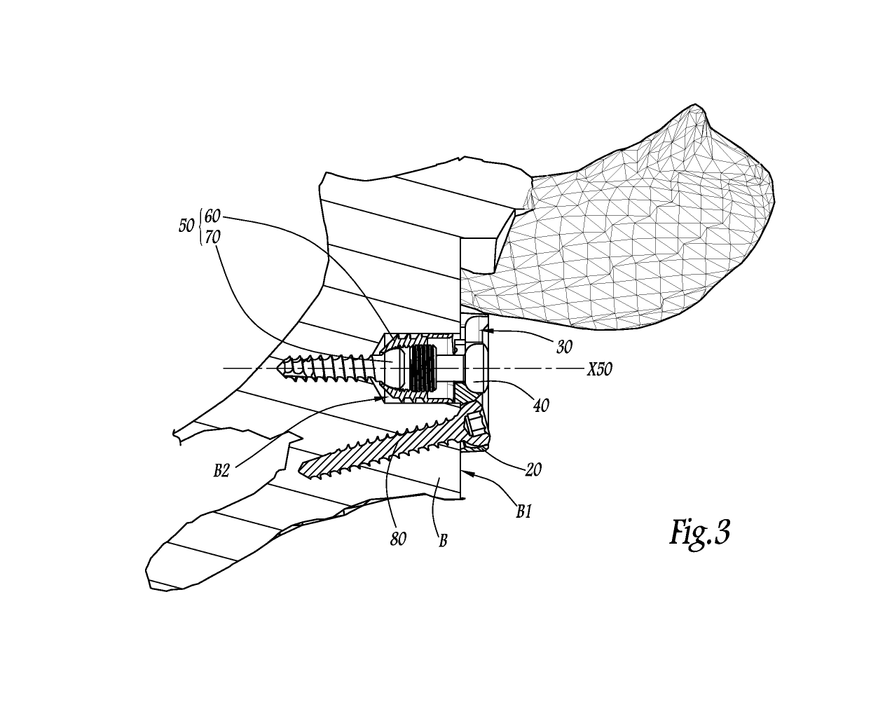

FIGS. 1-7 illustrate a glenoid implant 1 that is adapted to be implanted in the glenoid B of the scapula of a patient (the glenoid B is shown only in FIG. 3, and the humerus is not illustrated for the sake of simplification). The glenoid B includes a bone preparation which replaces the original glenoid cavity. The bone preparation includes an abutment surface B1 and an anchoring cavity B2. The surface B1 is substantially planar and may be formed, for example, by milling. The cavity B2 opens from the glenoid B and may be at least partially surrounded by the abutment surface B1.

Herein, in order to provide a spatial reference system, the term “rear side” of the implant 1 refers to the elements that face towards the glenoid B and “front side” of the implant 1 refers to the elements that face away from the glenoid B when the implant 1 is implanted in the glenoid B. In anatomical terms, therefore, the rear side is orientated in the medial direction and the front side is orientated in the lateral direction of the body of the patient.

The implant 1 includes an articular body 10 that is adapted to articulate with a humeral component (for example, the anatomical head of the humerus or a portion of a humeral implant). The implant 1 also includes a plate 20 (also referred to as a “base” or “base plate”) for supporting the articular body 10, which facilitates precisely positioning the articular body 10 relative to the glenoid B. The implant 1 also includes an adjustment pin 40, an anchoring mechanism 50 (also referred to as an “anchor”) including an insert 60 and a screw 70, and a securing screw 80. The insert 60 and the screw 70 are adapted to be permanently fixed to the glenoid B and, more specifically, fixed in the anchoring cavity B2 and extending along an anchoring axis X50. The pin 40 and the screw 80 form a securing mechanism that facilitates securing the plate 20 in position relative to the anchoring mechanism 50 and the glenoid B. The plate 20 includes a groove 30 (also referred to as a “slot”) for receiving the pin 40. The groove 30 and the pin 40 form an adjustment mechanism that facilitates adjusting the position of the plate 20 before it is selectively secured (both rotatably about the anchoring axis X50 and translatably relative to the anchoring axis X50) by the securing mechanism (that is, in some embodiments, the adjustment pin 40 and the securing screw 80). In some embodiments, the position of the plate 20 is selectively adjustable to permit the articular body 10 to be located at a desired position relative to the glenoid (for example, a relatively superior position or a relatively inferior position).

The elements 10, 20, 40, 60, 70 and 80 of the implant 1 may include one or more metals, metal alloys, ceramic materials or biocompatible polymers. The elements 10, 20, 40, 60, 70, and 80 may include, for example, stainless steel, titanium or titanium alloy, cobalt/chromium alloy, pyrocarbon, PEEK or any other suitable materials. Furthermore, the elements 10, 20, 40, 60, 70, and 80 may be subjected to any suitable local or non-local processing or covering operations.

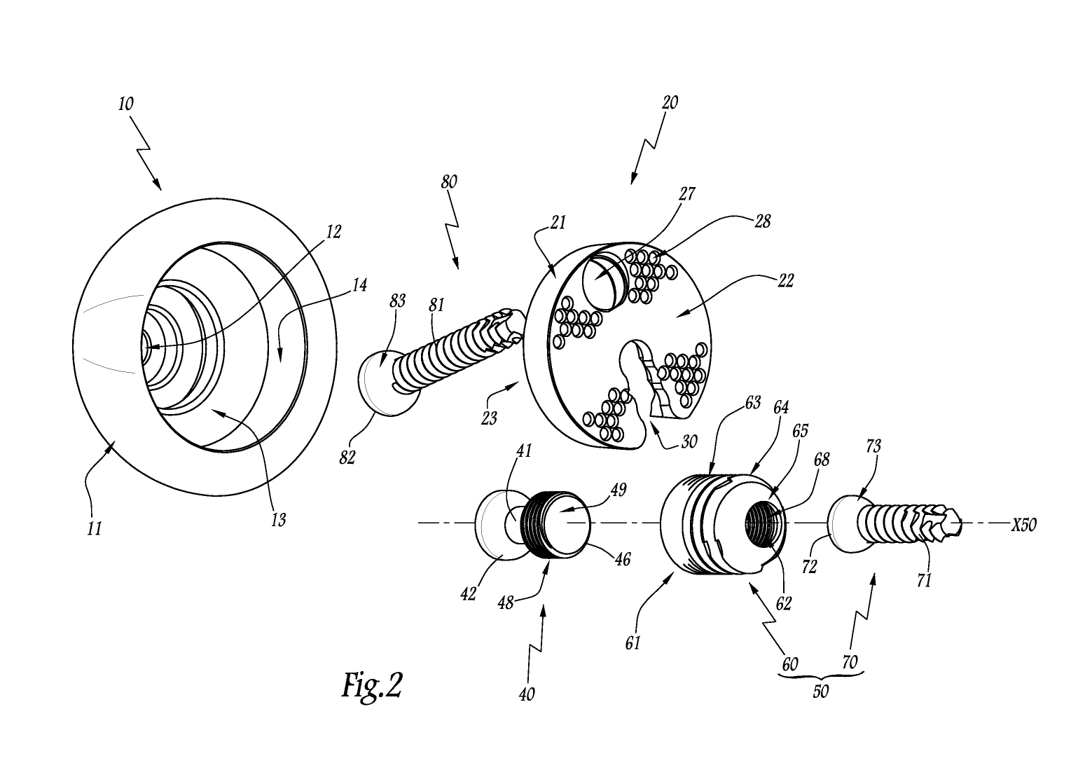

In the example of the figures, the implant 1 has a reversed configuration. As such, the articular body 10 is in the form of a hemispherical dome. The body 10 includes an outer articular surface 11 that has a convex spherical shape and an opening at the top 12. The body 10 also includes an internal cavity 13. In some embodiments, the internal cavity 13 includes a frustoconical surface 14 (see FIG. 2) for fixing to the plate 20. In some embodiments, the surface 14 may have a cylindrical profile. The opening 12 connects the external surface 11 and the internal cavity 13. The opening 12 permits the surgeon to access the pin 40 and the screw 80 when the body 10 is secured to the plate 20.

The plate 20 includes an outer frustoconical surface 21 that connects a rear side 22 and a front side 23 of the plate 20. The outer surface 21 has a greater diameter at the rear side 22 than at the front side 23. That is, the outer surface 21 tapers radially outwardly proceeding from the front side 23 to the rear side 22. The outer surface 21 engages the surface 14 of the articular body 10 in order to secure the articular body 10 relative to the glenoid B. At the front side 23, the plate 20 includes a front edge 24 which defines a front recess 25. The plate 20 includes an opening 27 that extends through the plate 20 from the front side 23 to the rear side 22. The opening 27 also receives the screw 80. The plate 20 includes holes 28 which are distributed on the rear side 22 to form an irregular surface on the rear side 22 and thereby promote bone regrowth. Besides the holes 28, the plate 20 has, at the rear side 22, a substantially planar surface suitable for abutting against the abutment surface B1 of the glenoid B.

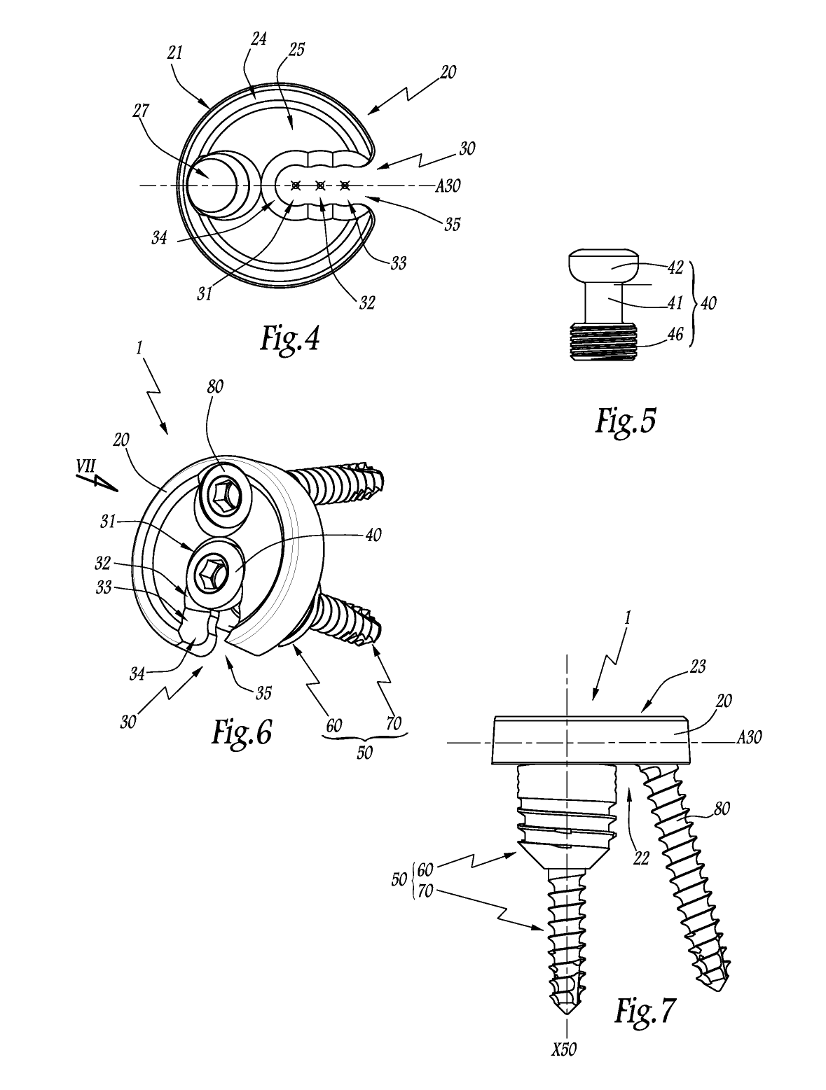

The groove 30 formed in the plate 20 generally extends along a groove axis A30, which is substantially perpendicular to the axis X50 when the plate 20 is secured to the glenoid B. The groove 30 opens at the outer surface 21, at the rear side 22, and at the front side 23 of the plate 20. The groove 30 includes cavities 31, 32 and 33 (see FIG. 4) which define separate positions for rotatably adjusting the plate 20. That is, the cavities 31-33 define different eccentric positions or eccentric axes about which the plate 20 may be selectively rotatably adjusted.

An edge 34 is formed around the cavities 31-33 at the front side 23 of the plate 20. As such, the cavities 31-33 are relatively narrow at the rear side 22 and relatively wide at the front side 23 of the groove 30. The edge 34 extends as a circular arc around the center of each cavity 31-33 and has, at the front side 23, a concavity that receives the pin 40. The cavity 31 is nearer to the center of the plate 20 and the recess 25 relative to the outer surface 21. The cavity 33 is formed near the outer surface 21 and the edge 24. The cavity 32 is formed between the cavities 31 and 33 in the recess 25. Along the axis A30, the groove 30 terminates at the end of the cavity 31 and includes a transverse opening 35 defined near the cavity 33 and the outer surface 21.

The pin 40 facilitates adjustment of the position of the plate 20 relative to the anchoring mechanism 50. The pin 40 includes a cylindrical rod 41 that connects a head 42 to a threaded base 46. The head 42 includes a curved rear surface 43 and an opening or hole 44, which may have a hexagonal shape, for receiving a tool (not shown), such as a surgical screwdriver. The rod 41 extends between the curved rear surface 43 of the head 42 and a front annular planar surface 47 of the base 46. The base 46 further includes a threaded surface 48 and a rear planar surface 49. The threaded base 46 may be coupled to (that is, screwed into) the anchoring mechanism 50 by inserting a tool, such as a surgical screwdriver, into the hole 44 and rotating to the tool to rotate the pin 40 about the anchoring axis X50. The head 42 of the pin 40 may be received in one of any of the cavities 31-33 to secure the plate 20 relative to the glenoid B in a position defined by the cavity 31, 32, or 33. As such and as described in further detail below, the pin 40 defines, in part, both to the adjustment mechanism and the securing mechanism.

The anchoring mechanism 50 is defined by the insert 60 and the screw 70 and extends along the anchoring axis X50. The insert 60 and the screw 70 are secured in the anchoring cavity B2. In some embodiments that are not depicted, the insert 60 and the screw 70 are formed as a single component. In some embodiments, the axis X50 and the anchoring mechanism 50 are substantially perpendicular to the abutment surface B1.

The insert 60 generally has a hollow-cylindrical shape with a circular cross-section. The insert 60 includes a front opening 61 and a rear opening 62. The insert 60 facilitates centering the implant 1 in the cavity B2 of the glenoid B. The insert 60 includes a raised external surface 63, an external thread 64, a rear frustoconical surface 65, an internal surface 66, an internal shoulder 67, an internal thread 68 for coupling to the pin 40, and an internal surface 69. In some embodiments, the anchoring axis X50 is defined by the insert 60 as opposed to the screw 70. In particular, the anchoring axis X50 may be defined by the internal thread 68 for coupling to the pin 40.

In some embodiments, the external surface 63 of the insert 60 includes raised portions that provide surface roughness to facilitate mechanically coupling the insert 60 to the internal walls of the anchoring cavity B2. The external thread 64, which may be self-tapping, facilitates anchoring the insert 60 to the glenoid B by screwing the insert into the cavity B2. In some embodiments and depending on the depth to which the insert 60 is inserted in the cavity B2, the rear surface 65 may abut the bottom of the cavity B2.

In some embodiments, the internal surface 66 (see FIG. 1) has an oval shape and is adapted to receive a tool, which may abut the shoulder 67, to facilitate screwing the insert 60 into the cavity B2. In some embodiments, the surface 66 may have other shapes. For example, the surface 66 may have a polygonal shape, for example, a hexagonal shape or a cylindrical shape. In such embodiments, the tool for screwing the insert 60 into the cavity B2 may engage the thread 68. When the insert 60 is positioned in the glenoid B, the thread 68 is able to receive the threaded base 46 of the pin 40, specifically the thread 48. The internal surface 69 faces towards the front opening 61 and abuttingly receives the screw 70.

The anchoring screw 70 includes a threaded body 71 and a screw head 72. The screw head 72 includes a curved rear surface 73 and a front opening or hole 74, which may have a hexagonal shape. The rear surface 73 abuts the internal surface 69 of the insert 60 when the threaded body 71 of the screw 70 is coupled to the glenoid B. The front hole 74 is adapted to receive a tool (not shown), such as a surgical screwdriver, to facilitate screwing the screw 70 into the glenoid B. In some embodiments, the axis of the screw 70 may be aligned with the anchoring axis X50 defined by the insert 60. In some embodiments, when the screw 70 is screwed into the glenoid B, the axis of that screw 70 may be inclined relative to the anchoring axis X50.

The securing screw 80 includes a threaded body 81 and a screw head 82. The screw head 82 includes a curved rear surface 83 and a front opening or hole 84, which may have a hexagonal shape. The opening 84 is adapted to receive a tool (not shown), such as a surgical screwdriver, to facilitate screwing the threaded body 81 of the screw 80 into the glenoid B. The rear surface 83 abuts the edges of the opening 27 of the plate 20 when the threaded body 81 of the screw 80 is coupled to the glenoid B. In some embodiments, the screw 80 is a “multidirectional” screw and the opening 27 is shaped to receive the screw 80 along multiple axes relative to the plate 20 and the glenoid B. As such, a surgeon may position the screw 80 along any of various desirable axes relative to the plate 20 and the glenoid B. The screw 80, when received in the opening 27 of the plate 20, secures the plate 20 relative to the glenoid B. In particular, the screw 80 rotatably secures the plate 20 about the anchoring axis X50 of the anchoring mechanism 50 implanted in the glenoid B.

In some embodiments that are not depicted, the multidirectional securing screw 80 may be replaced or supplemented by an expansion-type securing mechanism. For example, such a securing mechanism may include a washer that radially expands in the opening 27 while rotating a screw in the washer and into bone.

In some embodiments, a method for implanting the implant 1 is as follows.

The surgeon first prepares the abutment surface B1 and the anchoring cavity B2. The surgeon may prepare the surface B1 before or after the cavity B2. As described above, the implant 1 may include either a reversed articular body 10 or an anatomical articular body. An anatomical articular body may be replaced by a reversed articular body 10 during a later revision procedure.

The cavity B2 may be prepared as a hole that has a diameter that is slightly smaller than the outer diameter of the insert 60, specifically the thread 64 of the insert 60. The insert 60 is then screwed in the cavity B2 via a tool (not shown) that includes a distal end or tip adapted to couple to the internal surface 66 of the insert 60. Another hole is then prepared in the glenoid B that has a diameter that is slightly smaller than the diameter of the threaded body 71 of the screw 70. In some embodiments, the hole is formed by inserting a drill bit (not shown) through the openings 61 and 62 of the insert 60 and perpendicularly to the glenoid surface B1. The drill bit may be guided by an additional tool (not shown) that is positioned inside the insert 60. The anchoring screw 70 is subsequently screwed into the hole and into the insert 60 via a tool (not shown) that is inserted into the hole 74 of the screw 70. Ultimately, the rear surface 73 of the screw 70 abuts the internal surface 69 of the insert 60. In some embodiments and as shown in the Figures, the thread 64 of the insert 60 is “right-handed” and the insert 60 is screwed in a counter-clockwise direction to secure the component to bone. In contrast, the thread 71 of the screw 70 is “left-handed” and the screw 70 is screwed in a clockwise direction to secure the component to bone. As such, once the insert 60 and the screw 70 are positioned in the cavity B2, the anchoring mechanism 50 is rotatably secured and inhibited from being unscrewed. In some embodiments, the insert 60 is positioned to provide a small space between the front opening 61 of the insert 60 and the mouth of the cavity B2. Such a construction permits milling of a new surface B1 without milling the insert 60.

In a revision procedure for the glenoid B, for example, to replace an anatomical articular body with a reversed articular body 10, the previously-formed cavity B2 and the previously-implanted anchoring mechanism 50 may be used. However, the surface B1 may be formed as a replacement for the previous surface of the glenoid B. The surface B1 may be inclined relative to a plane perpendicular to the previously-defined anchoring axis X50.

For either, a first procedure or a revision procedure, the pin 40 is inserted into the insert 60 via the opening 61. A tool (not shown) is then inserted into the hole 44 of the head 42 of the pin 40 to screw the threaded base 46 of the pin 40 into the thread 68 of the insert 60. After this action, the rod 41 and the head 42 of the pin 40 extend out of the anchoring cavity B2 and past the abutment surface B1. The plate 20 may then be positioned about the pin 40 (that is, such that the groove 30 receives the rod 41), by translating plate 20 across the abutment surface B1. In some embodiments, the edge 34 of the groove 30 is dimensioned to slide against the external surface of the rod 41. Due to the groove 30, the plate 20 may simultaneously pivot and translate relative to the pin 40 and the anchoring axis X50.

As such, the groove 30 and the pin 40 define an adjustment mechanism for selectively adjusting the position of the plate 20, before it is secured in position by the pin 40 and the screw 80, in rotation about the anchoring axis X50 and in translation transversely relative to the anchoring axis X50. Stated another way, the groove 30 permits transverse translational adjustment of the plate 20 relative to the axis X50 and rotational adjustment of the plate 20 about the axis X50. For a given angular orientation of the plate 20 and the axis A30 about the axis X50, translation of the plate 20 is permitted, and each cavity 31-33 defines a different transverse position of the plate 20 relative to the axis X50. Stated another way, each cavity 31-33 defines a different eccentric axis of the plate 20 for rotation of the plate 20 relative to the glenoid B.

The groove 30 and the pin 40 define an adjustment mechanism for pivotably or tiltably adjusting the position of the plate 20 relative to the anchoring axis X50. This adjustment is facilitated by a ball-type connection between the head 42 of the pin 40 and one of any of the cavities 31, 32 or 33 of the groove 30. As such, if the surface B1 is not exactly perpendicular to the axis X50, the plate 20 may pivot relative to that axis X50 to abut the glenoid surface B1.

After making one or more of the adjustments described above, the plate 20 is secured relative to the anchoring mechanism 50, the anchoring axis X50, and the glenoid B by the pin 40 and the screw 80. Specifically, the pin 40 is further screwed into the insert 60, and the surface 43 of the pin 40 abuts the edge 34 of the groove 30 and is received in one of the cavities 31-33. This action secures the plate 20 relative to the insert 60.

In some embodiments, the pin 40 is sufficient to secure the plate 20 relative to the anchoring mechanism 50, the axis X50, and the glenoid B. In some embodiments, both the pin 40 and the screw 80 are used to secure the plate 20 relative to the anchoring mechanism 50, the axis X50, and the glenoid B. That is, in some embodiments, the securing mechanism includes the pin 40 and, in some embodiments, the securing mechanism includes the pin 40 and the screw 80.

To couple the screw 80 to the implant 1 and the glenoid B, another hole is then formed in the glenoid B that has a diameter that is slightly smaller than the diameter of the threaded body 81 of the screw 80. The hole may be formed by passing a drill bit (not shown) through the opening 27 of the plate 20 in a non-perpendicular direction relative to the surface B1. The drill bit may be guided by an additional tool or guide (not shown) that is temporarily positioned in the opening 27. After forming the hole, the securing screw 80 is screwed into the hole and into the opening 27. The screw 80 may be driven using a tool (not shown) that is received in the opening 84 of the screw 80.

The articular body 10 is then secured to the plate 20 by, for example, impacting the body 10 onto the plate 20. Thus, in some embodiments, accurate positioning of the plate 20 facilitates accurate positioning of the articular surface 11 of the body 10 relative to the glenoid B. In some embodiments, the above method reduces the risk of disassembly of the components of the implant 1. After the implantation procedure, the body 10 is articulated with the humerus to provide either the reversed shoulder prosthesis type in the example of FIG. 3 or of the anatomical shoulder prosthesis type when the articular body has an anatomical configuration.

In some embodiments, the implant 1 may be provided as part of a surgical kit according the invention. In some embodiments, the surgical kit includes at least one articular body and at least one plate, such as the body 10 and the plate 20 illustrated in the example of FIGS. 1 to 7. In some embodiments, the surgical kit includes a plurality of articular bodies and/or a plurality of plates, such as the plates 20, 120, 220, 320, 420 and 520 described above and below.

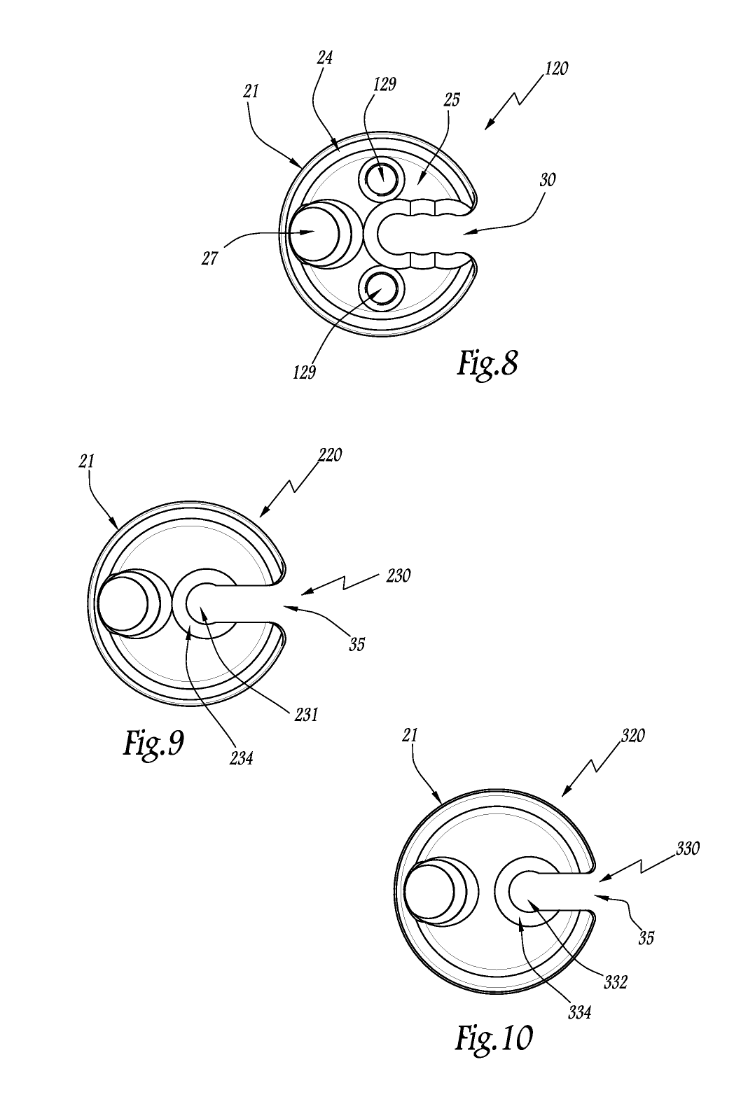

FIGS. 8, 9 and 10 illustrate plates 120, 220 and 320 according to embodiments of the invention.

Some of the features of the plates 120, 220 and 320 are similar to those of the plate 20 and have the same reference numerals. For brevity, only different and additional features relative to the plate 20 are described in detail below.

As shown in FIG. 8, the plate 120 includes two additional through-openings 129 disposed on opposite sides of the groove 30. The openings 129 are adapted for receiving additional securing elements, such as screws (for example, the screw 80) or any other suitable securing elements.

As shown in FIGS. 9 and 10, the plates 220 and 320 each include a groove 230 and 330, respectively, that differs from the groove 30. In these embodiments, each groove 230 and 330 includes a single cavity 231 or 332, respectively. The cavities 231 and 332 are bounded by local edges 234 and 334, respectively. The grooves 230 and 330 form, together with the pin 40, adjustment mechanisms for selectively adjusting the position of the plate 220 or 320 before it is secured by the securing mechanism (in terms of rotation about the axis X50, translation relative to the axis X50, and pivoting relative to the axis X50). The cavities 231 and 332 define different translational offsets relative to the outer surface 21 for receiving the articular body 10.

In some embodiments, the plates 220 and 320 are provided in a surgical kit according to the invention. As such, a surgeon may select one of the plates 220 and 320 based on the desired translation adjustment relative to the anchoring axis X50. The two plates 220 and 320 are also selectively rotatably adjustable about the axis X50 and selectively transversely translatably adjustable relative to the axis X50. However, the plates 220 and 320 are adjustable to different configurations due to the different positioning of the cavities 231 and 332. In addition the plates 220 and 320 are also selectively pivotably adjustable, albeit to different configurations due to the different positioning of the cavities 231 and 332.

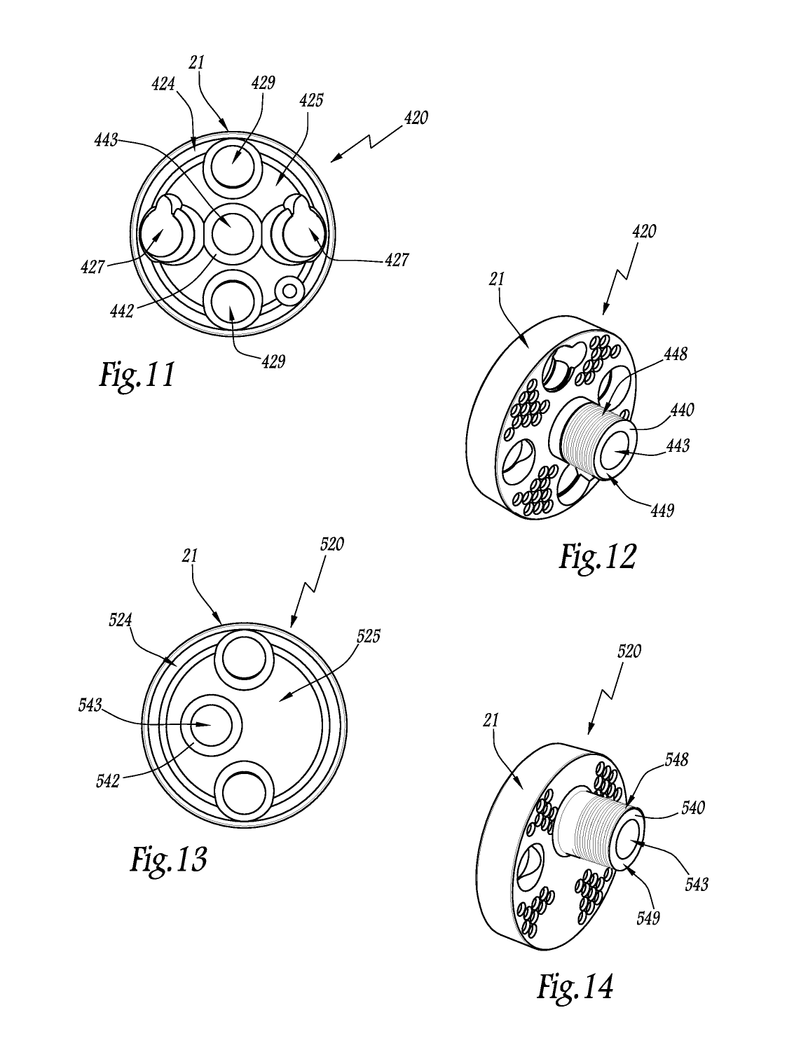

FIGS. 11 to 14 illustrate plates 420 and 520 according to embodiments of the invention, and which may be provided in a surgical kit according to embodiments of the invention.

Some features of the plates 420 and 520 are similar to those of the plate 20, for example, the outer surface 21 that receives the articular body 10. For brevity, only the differences relative to the plate 20 are described in detail below.

The plates 420 and 520 lack a groove for adjustment. Instead, the plates 420 and 520 include pins 440 and 540, respectively, that facilitate positional adjustment. The pins 440 and 540 are integrally or monolithically formed in the plates 420 and 520, respectively, and are adapted to couple to the anchoring mechanism 50 of the implant 1.

As shown in FIGS. 11 and 12, the plate 420 includes an edge 424 and a cavity 425 that are located on the front side of the plate 420. The plate 420 also includes two through-openings 427 and two through-openings 429 that extend from the front side to a rear side of the plate 420. The openings 427 are generally similar to the opening 27 and the openings 429 are generally similar to the openings 129. In some embodiments, the openings 427 and 429 extend through both the edge 424 and the cavity 425 and are disposed 90 degrees apart from each other about the pin 440.

In some embodiments, the pin 440 has a cylindrical cross-sectional shape that is centered relative to the outer surface 21 of the plate 420. The pin 440 includes an annular front edge 442 that is located in the recess 425. The pin 440 extends from the annular front edge 442, through the plate 420, and to a rear annular edge 449. The pin 440 also includes a cylindrical bore 443 that extends through the plate 420 between the edges 442 and 449. The pin 440 has a greater height dimension at the rear side of the plate 420 compared to the front side of the plate 420. At the rear side of the plate 420, the pin 440 includes a thread 448 that is adapted to couple to the anchoring mechanism 50 of the implant 1. The pin 440 defines an adjustment mechanism for rotatably adjusting the plate 420 relative to the anchoring axis X50. When the orientation of the plate 420 is adjusted relative to the axis X50, the positions of the holes 427 and 429 are adjusted relative to the axis X50.

As shown in FIGS. 13 and 14, the pin 540 is similar to the pin 440 and includes the many of the same features. In FIGS. 13 and 14, such features have reference numerals increased by 100 compared to those of FIGS. 11 and 12. For example, the pin 540 includes edges 542 and 549, a bore 543, and a thread 548, which is adapted to couple to the anchoring mechanism 50 of the implant 1. The plate 520 further includes an edge 524 and a cavity 525 that are located at the front side and two through-openings that are identical to the openings 429.

In some embodiments, the pin 540 is off-center relative to the outer surface 21 of the plate 520. The pin 540 defines an adjustment mechanism for rotatably adjusting the plate 520 relative to the anchoring axis X50. When the orientation of the plate 520 is adjusted relative to the axis X50, the positions of the openings that receive securing screws are adjusted. In addition, the position of the outer surface 21 of the plate 520 is also adjusted due to the off-center position of the pin 540.

In some embodiments that are not depicted, the pins 420 or 520 may couple to the anchoring mechanism 50 via different components, for example, with a rack type device, an expansion element, a snap ring, or the like.

The positions of each of the plates 420 or 520 may be rotatably adjusted before being secured by the securing mechanism. When the plates 420 and 520 are provided in a surgical kit according to embodiments of the invention, a surgeon may select one of the plates 420 and 520 based on the desired adjustment of the outer surface 21 relative to the axis X50. Thus, the two plates 420 and 520 are rotatably selectively adjustable about the axis X50. In addition, the plates 420 and 520 are adjustable to a different set of configurations due to the off-center position of the pin 540 of the plate 520.

The implant 1 and/or the plates may take different forms without departing from the scope of the invention. For example, the articular body, the anchoring mechanism, the securing mechanism and/or the adjustment mechanism may take different forms than the exemplary embodiments that are shown and described.

In addition, the features of the different embodiments may be, in their entirety or for some of them, combined with each other.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A glenoid implant for a shoulder prosthesis, the glenoid implant being adapted to be implanted in the glenoid of a patient and comprising:
an articular body for articulating the glenoid implant with a humeral component;
a plate for supporting the articular body;
an anchoring mechanism for anchoring the glenoid implant along an anchoring axis and adapted to be secured to the glenoid; and
a securing mechanism for securing the plate in position relative to the anchoring mechanism and the glenoid;
an adjustment mechanism for selectively adjusting the position of the plate, before it is secured in position by the securing mechanism, in rotation about the anchoring axis and in translation transversely to the anchoring axis.

2. The glenoid implant according to claim 1, wherein the adjustment mechanism includes a ball-type connection for selectively tiltably adjusting the position of the plate, before it is secured in position by the securing mechanism, relative to the anchoring axis.

3. The glenoid implant according to claim 1, wherein the adjustment mechanism includes a groove formed in the plate, the groove extending in a direction transverse to the anchoring axis and facilitating adjustment of the position of the plate transversely to the anchoring axis and independently of rotation of the plate about the anchoring axis.

4. The glenoid implant according to claim 3, wherein the groove includes at least two cavities that each define a distinct position of the plate transverse to the anchoring axis and independently of rotation of the plate about the anchoring axis.

5. The glenoid implant according to claim 1, wherein the adjustment mechanism includes a pin that cooperates with the anchoring mechanism and the plate to adjust the position of the plate relative to the anchoring axis.

6. The glenoid implant according to claim 1, wherein the articular body has one of an anatomic and a reversed configuration.

7. A glenoid implant for a shoulder prosthesis, the glenoid implant being adapted to be implanted in the glenoid of a patient and comprising:
an anchor adapted to be secured to the glenoid, the anchor defining an anchoring axis;
a base including a slot adapted to relatively movably receive the anchor, the slot and the anchor facilitating rotational adjustment of the base relative to the anchor about the anchoring axis and translational adjustment of the base relative to the anchor in a direction transverse to the anchoring axis; and
an articular body adapted to be supported by the base and to articulate with a humeral component.

8. The glenoid implant according to claim 7, wherein the base further includes a hole, and further comprising a screw adapted to be received by the hole and secured to the glenoid to inhibit rotational and translational adjustment of the base relative to the anchor.

9. The glenoid implant according to claim 8, wherein the screw is a multidirectional screw.

10. The glenoid implant according to claim 7, wherein the slot includes a plurality of cavities adapted to receive the anchor, each of the cavities defining an eccentric axis about which the base is rotatably adjustable relative to the anchor.

11. The glenoid implant according to claim 7, wherein the anchor includes:
an insert adapted to be secured to the glenoid; and
an adjustment pin adapted to be detachably coupled to the insert, the adjustment pin being adapted to be relatively movably received by the slot, the slot and the adjustment pin facilitating rotational adjustment of the base relative to the anchor about the anchoring axis and translational adjustment of the base relative to the anchor in the direction transverse to the anchoring axis.

12. The glenoid implant according to claim 11, wherein the anchor further includes a screw adapted to engage the insert and be secured to the glenoid.
